(19) United States
(12) Patent Application Publication
(10) Pub. No.: US 2023/0044107 A1
(43) Pub. Date: Feb. 9, 2023

(54) WATER IN OIL TYPE EMULSION COSMETIC

(71) Applicant: Shiseido Company, Ltd., Chuo-ku, Tokyo (JP)

(72) Inventors: Ryo FURUKAWA, Tokyo (JP); Ayumu ENOMOTO, Tokyo (JP)

(73) Assignee: Shiseido Company, Ltd., Chuo-ku, Tokyo (JP)

(21) Appl. No.: 17/777,325

(22) PCT Filed: Nov. 6, 2020
(86) PCT No.: PCT/JP2020/041482

§ 371 Date: May 17, 2022

(30) Foreign Application Priority Data
Nov. 18, 2019 (JP) 2019-207990

(51) Int. Cl. A61K 8/06; A61K 8/37; A61K 8/891; A61Q 17/04; A61K 8/73

(52) U.S. Cl. CPC A61K 8/06; A61K 8/37; A61K 8/891; A61Q 17/04; A61K 8/731

(57) ABSTRACT

To provide a water in oil type emulsion cosmetic capable of obtaining a smooth and fresh sensation of use, and has superior emulsion stability. A water in oil type emulsion cosmetic contains (A) cetyl PEG/PPG 10-1 dimethicone; (B) silicone wax; (C) polyethylene glycol; and (D) a polar oil.

### CROSS REFERENCE TO RELATED APPLICATIONS

The present application is a National Phase Entry of PCT International patent Application No. PCT/JP2020/041482 filed on Nov. 6, 2020, which claims priority to Japanese Patent Application No. 2019-207990 filed on Nov. 18, 2019.

### TECHNICAL FIELD

The present disclosure is related to a water in oil type emulsion cosmetic.

### BACKGROUND ART

There are various types of emulsion types for emulsion bases, such as the oil in water type, the water in oil type, composites thereof, etc. Among these emulsion types, it is known that water in oil type emulsion cosmetics exhibit superior skin protecting effects, softening effects, moisturizing effects, etc. compared to oil in water type emulsion cosmetics.

However, if an oil gelling agent such as an organic modified clay mineral, for example, is blended into a water in oil type emulsion cosmetic, there are cases in which usability will deteriorate, by the cosmetic becoming difficult to spread on the skin, a sensation of freshness diminishing, the cosmetic becoming difficult to take out of a container, etc. In order to solve these problems, Japanese Patent No. 6277307, for example, discloses a water in oil type liquid emulsion composition, in which 55 to 80% by mass of water is contained in cetyl dimethicone copolyol and poly glyceryl diisostearate. This water in oil type liquid emulsion composition obtains a favorable sensation of freshness when applied, and a usability without stickiness by a large amount of water being blended therein.

In addition, International Patent Publication No. WO 2018/216696 discloses that a water in oil type cosmetic that contains an oil solution having a melting point of less than or equal to 20° C., silicone gel, a modified silicone surfactant having a hydrophilic-lipophilic balance with a value of less than 6, inorganic particles, water, and an emulsifying assisting agent has superior emulsifying properties and emulsion stability, as well as a favorable sensation of water outflow when applied to the skin, and that unevenness in the applied cosmetic is reduced.

### BACKGROUND ART DOCUMENTS

### Summary

The water in oil type liquid emulsion composition disclosed in Japanese Patent No. 6277307 obtains a sensation of use without stickiness by blending a large amount of water therein. However, there are aspects of usability such as a desire for the skin to appear fuller and smoother and a desire for skin protecting properties to be improved. There is also an aspect of a desire for an oil soluble effective component such as an emollient oil, an oil-soluble medicament, etc. to be efficiently delivered onto the skin. Further, there is a viewpoint that an SPF (Sun Protection Factor) value to be increased. Based on the above, there are cases in which it is desired for a large amount of a polar oil to be blended. However, it is difficult for oils having high polarity to cause gelling and water which had been emulsified may separate. That is, it is difficult to achieve stability if a large amount of a polar oil is blended. In Japanese Patent No. 6277307, cetyl dimethicone copolyol and poly glyceryl diisostearate, which are emulsion promoting agents, are combined to obtain stability, etc. at high temperatures. However, a preparation of types of oils have a greater degree of versatility as formulations.

Meanwhile, the cosmetic of International Patent Publication No. WO 2018/216696 contains greater than or equal to 60% by mass of silicone oil in an oil solution to stably emulsify the inorganic particles. However, if a large amount of silicone oil is blended, there are cases in which a skin softening effect cannot be sufficiently obtained, cases in which it becomes difficult to obtain a stable water in oil type emulsion cosmetic, and cases in which stability will deteriorate at high temperatures.

The present disclosure has been developed in view of the foregoing circumstances. The present disclosure provides a water in oil type emulsion cosmetic which is smooth, exhibits a fresh sensation of use, and has superior emulsion stability.

The water in oil type emulsion cosmetic of the present disclosure contains:

(A) cetyl PEG/PPG 10-1 dimethicone;

(B) silicone wax;

(C) polyethylene glycol; and

(D) a polar oil.

It is preferable for the water in oil type emulsion cosmetic of the present disclosure to contain the (D) polar oil in an amount within a range from 6 to 40% by mass with respect to the total amount of the cosmetic.

It is preferable for the molecular weight of the (C) polyethylene glycol to be within a range from 6000 to 20000.

It is preferable for the water in oil type emulsion cosmetic of the present disclosure to further contain (E) cellulose gum or a polysaccharide thickener.

In the water in oil type emulsion cosmetic of the present disclosure, the content of dimethicone and/or cyclic silicone oil may be less than or equal to 10% by mass with respect to the total amount of the cosmetic.

The water in oil type emulsion cosmetic of the present disclosure contains:

(A) cetyl PEG/PPG 10-1 dimethicone;

(B) silicone wax;

(C) polyethylene glycol; and

(D) a polar oil.

Therefore, the water in oil type emulsion cosmetic is that which is smooth, exhibits a fresh sensation of use, and has superior emulsion stability.

### DESCRIPTION OF EMBODIMENTS

The water in oil type emulsion cosmetic of the present disclosure contains:

(A) cetyl PEG/PPG 10-1 dimethicone;

(B) silicone wax;

(C) polyethylene glycol; and

(D) a polar oil.

Each of the components will be described in detail below. Note that in the present specification, PEG is an abbreviation for polyethylene glycol, PPG is an abbreviation for polypropylene glycol, EO is an abbreviation for ethylene oxide, PO is an abbreviation for propylene oxide, POE is an abbreviation for poly oxy ethylene, POP is an abbreviation for poly oxy propylene, VP is an abbreviation for vinyl pyrrolidone, and DPG is an abbreviation for dipropylene glycol.

### (A) Cetyl PEG/PPG 10-1 Dimethicone

The water in oil type emulsion cosmetic of the present disclosure contains cetyl PEG/PPG 10-1 dimethicone. Commercially available products such as ABIL EM90 and EMI 180 (Evonik Industries AG, Co.) and KF-6048 (Shin Etsu Chemical Industries, K. K.), for example, may be employed as the (A) cetyl PEG/PPG 10-1 dimethicone.

One type of the (A) cetyl PEG/PPG 10-1 dimethicone may be utilized, or two or more types may be utilized in combination.

The content of the (A) cetyl PEG/PPG 10-1 dimethicone is within a range from 0.5 to 10% by mass, and preferably within a range from 1 to 5% by mass with respect to the total amount of the cosmetic. By the content of the (A) cetyl PEG/PPG 10-1 dimethicone being greater than or equal to 0.5% by mass, emulsion stability can become further favorable. By the content of the (A) cetyl PEG/PPG 10-1 dimethicone being less than or equal to 10% by mass, a fresher sensation of use can be obtained.

### (B) Silicone Wax

The (B) silicone wax to be employed in the water in oil type emulsion cosmetic of the present disclosure is not particularly limited as long as it is a raw material which is generally blended into cosmetics. Examples of such silicone waxes include highly polymerized methyl polysiloxanes such as highly polymerized dimethyl polysiloxane, highly polymerized methyl phenyl siloxane, highly polymerized methyl vinylpolysiloxane, etc., highly polymerized amino modified methyl polysiloxane, alkyl modified silicones (stearyl dimethicone, alkyl (C30-C45) methicone, etc., for example) polyamide modified silicones, long chain alkoxy modified silanes (stearoxy trimethyl silane, etc.), etc. One type or a combination of two or more types of these silicone waxes may be employed as necessary.

Examples of the (B) silicone waxes which are commercially available include silicone wax AMS-C30 (Dow Chemical Japan K. K./Dow Toray K. K.), etc.

The amount of the (B) silicone wax to be blended is within a range from 0.1 to 10% by mass, and preferably within a range from 0.2 to 5% by mass with respect to the total amount of the cosmetic. By the amount of the (B) silicone wax being greater than or equal to 0.1% by mass, emulsion stability can become more favorable. By the amount of the (B) silicone wax being less than or equal to 10% by mass, a smoother and fresher sensation of use can be obtained.

### (C) Polyethylene Glycol

The water in oil type emulsion cosmetic of the present disclosure contains (C) polyethylene glycol.

It is preferable for the molecular weight of the (C) polyethylene glycol to be within a range from 6000 to 20000, and more preferably within a range from 6000 to 18000. By the molecular weight of the (C) polyethylene glycol being greater than or equal to 6000, stability at high temperatures can be further improved. By the molecular weight of the (C) polyethylene glycol being less than or equal to 20000, a fresher sensation of use can be obtained.

The amount of the (C) polyethylene glycol to be blended is preferably within a range from 0.5 to 5% by mass, and preferably within a range from 0.8 to 3% by mass with respect to the total amount of the cosmetic. By the amount of the (C) polyethylene glycol being greater than or equal to 0.5% by mass, a more stable water in oil type emulsion cosmetic can be obtained. By the amount of the (C) polyethylene glycol being less than or equal to 5% by mass, a fresher sensation of use can be obtained.

### (D) Polar Oil

The (D) polar is not particularly limited as long as it is that which is generally employed in cosmetics, medicaments, and food. Although the IOB value of the polar oil component is not particularly limited, it is preferable for the IOB value to be within a range from 0.05 to 0.80.

Note that the IOB value is an abbreviation for “Inorganic/Organic Balance” that represents the ratio of an inorganic value with respect to an organic value, and is an index that represents the degree of polarity of an organic compound. Specifically, the IOB value is represented as:

IOB value=Inorganic Value/Organic Value

Here, with respect to the “inorganic value” and the “organic value”, an “inorganic value” and an “organic value” are set according to each type of atom or functional group, such as an “organic value” of 20 for each carbon atom within a molecule and an “inorganic value” of 100 for each hydroxy group within a molecule, for example. The IOB value of the organic compound can be calculated by integrating the “inorganic value” and the “organic value” of all of the atoms and functional groups within the organic compound (refer to Fujita, Chemical Regions, Vol. 11, No. 10, pp. 719-725, 1957, for example).

Representative examples of the polar oil include ester oils and UV ray absorbers.

Specific examples of ester oils include tripropylene glycol dineopentanate, isononyl isononanoate, isopropyl myristate, cetyl octanoate, octyl dodecyl myristate, isopropyl palmitate, butyl stearate, hexyl laurate, myristyl myristate, decyl oleate, hexyl decyl dimethyl octanoate, cetyl lactate, myristyl lactate, lanolin acetate, isocetyl stearate, isocetyl isostearate, cholesteryl 12-hydroxy stearate, cetyl ethyl hexanate, ethylene glycol di-2-ethyl hexanoate, dipentaerythritol fatty acid ester, N-alkyl glycol monoisostearate, neopentyl glycol dicaprate, diisostearyl malate, glycerin di-2-heptylundecanoate, trimethylolpropane tri-2-ethyl hexanoate, trimethylolpropane triisostearate, penta erythrityl tetra-2-ethyl hexanoate, triethylhexanoin (glycerin tri-2-ethyl hexanoate), glycerin trioctanoate, glycerin triisopalmitate, trimethylolpropane triisostearate, cetyl 2-ethyl hexanoate, 2-ethyl hexyl palmitate, glycerin trimyristate, glyceride tri-2-heptylundecanoate, castor oil fatty acid methyl ester, oleyl oleate, acetoglyceride, 2-heptylundecyl palmitate, diisobutyl adipate, N-lauroyl-L-glutamate-2-octyl dodecyl ester, di-2-heptylundecyl adipate, ethyl laurate, di-2-ethyl hexyl sebacate, 2-hexyl decyl myristate, 2-hexyl decyl palmitate, 2-hexyl decyl adipate, diisopropyl sebacate, 2-ethyl hexyl succinate, triethyl citrate, etc.

There are a great number of oil based UV ray absorbers having high polarity which are generally employed in cosmetics, and the UV ray absorber to be employed is not particularly limited. Examples of such oil based UV ray absorbers include benzoic acid derivatives, salicylic acid derivatives, cinnamic acid derivatives, dibenzoylmethane derivatives, β, β-diphenyl acrylate derivatives, benzophenone derivatives, benzylidene camphor derivatives, phenyl benzo imidazole derivatives, triazine derivatives, phenyl benzotriazole derivatives, anthranyl derivatives, imidazoline derivatives, benzal malonate derivatives, and 4,4-diarylbutadiene derivatives. Specific examples and product names will be listed below. However, the UV ray absorber is not limited to those listed below.

Examples of benzoic acid derivatives include para-amino benzoic acid (PABA) ethyl, ethyl-dihydroxy propyl PABA, ethyl hexyl-dimethyl PABA (“Escalol 507” by ISP Chemicals, for example), glyceryl PABA, PEG-25-PABA (“Uvinal P25” by BASF Co., for example), diethyl amino hydroxy benzoyl benzoic acid hexyl (“Uvinal A Plus”, for example).

Examples of salicylic acid derivatives include homosalate (“Eusolex HMS” by Lona/EM Industries Co., for example), ethyl hexyl salicylate (“Neo Heliopan OS” by Harman & Reimer Co., for example), dipropylene glycol salicylate (“Dipsal” by Skel Co., for example), TEA salicylate (“Neo Heliopan TS” by Harman and Reimer Co., for example), etc.

Examples of cinnamic acid derivatives include octyl methoxycinnamate or ethyl hexyl methoxycinnamate (“Pulsol MCX” by Hoffmann-La Roche Co., for example), isopropyl methoxycinnamate, isoamyl methoxycinnamate (“Neo Heliopan E1000” by Herman and Reimer Co., for example), synnoxate, DEA methoxycinnamate, di isopropyl methyl silicate, glyceryl-ethyl hexanoate-di methoxycinnamate, di-(2-ethyl hexyl)-4′-methoxybenzalmalonate, etc. 4-tert-butyl-4′-methoxydibenzoylmethane (“Pulsol 1789”, for example) is an example of a dibenzoyl methane derivative.

Octocrylene (“Ubinal N539” by BASF, for example) is an example of a (3, (3-diphenyl acrylate derivative.

Benzophenone derivatives include benzophenone-1 (“Uvinal 400” by BASF Co.), benzophenone-2 (“Uvinal D50” by BASF Co., for example), benzophenone-3 or oxybenzone (“Uvinal M40” by BASF, for example), benzophenone-4 (“Uvinal MS40” by BASF Co., for example), benzophenone-5, benzophenone-6 (“Helisorb 11” by Norquay Co., for example), benzophenone-8 (“Spectra-Sorb UV-24” by American Cyanamide Co., for example), benzophenone-9 (“Uvinal DS-49” by BASF Co., for example), benzophenone-12, etc.

Examples of benzylidene camphor derivatives include 3-benzylidene camphor (“Mexoryl SD” by Chimex Co., for example), 4-methyl benzylidene camphor, benzylidene camphor sulfonate (“Mexoryl SL” by Chimex Co., for example), camphor benzalkonium methosulfate (“Mexoryl SO” by Chimex Co., for example), terephthaliledene di camphor sulfonate (“Mexoryl SX” by Chimex Co., for example), polyacrylamide methyl benzylidene camphor (“Mexoryl SW” by Chimex Co., for example), etc.

Examples of phenyl benzimidazole derivatives include phenyl benzimidazole sulfonate (“Eusolex 232” by Merck Co., for example), disodium phenyl dibenzo imidazole tetra sulfonate (“Neo Heliopan AP” by Herman & Reimer Co.), etc.

Examples of triazine derivatives include anisotriazine (“Tinosorb S” by Ciba Specialty Chemicals Co., for example), ethyl hexyl triazone (“Uvinal T150” by BASF Co., for example), diethyhexyl butamide triazone (“Uvasorb HEB” by Sigma 3V Co. for example), 2,4,6-tris (diisobutyl-4′-aminobenzalmaronate)-s-triazine, etc.

Examples of phenyl benzotriazole derivatives include drometrizole trisiloxane (“Silatrizole” by Rhodia Chimie Co., for example), methylene bis (benzo triazolyl tetramethyl butylphenol) (“Tinosorb M” by Ciba Specialty Chemicals Co.), etc.

Menthyl anthranilate (“Neo Heliopan MA” by Harman & Reimer Co., for example) is an example of an anthranyl derivative.

Ethyl hexyl dimethoxy benzylidene dioxoimidazolin propionate is an example of an imidazoline derivative.

Poly organo siloxane having a benzal malonate functional group (polysilicone-15 “Pulsol SLX” by DSM Nutrition Japan Co., for example) is an example of a benzal malonate derivative.

1,1-dicarboxy (2,2′-dimethyl propyl)-4,4-diphenyl butadiene is an example of a 4,4-diaryl butadiene derivative.

One type or a combination of two or more of the (D) polar oil may be utilized.

It is preferable for the amount of the (D) polar oil to be blended to be within a range from 6 to 40% by mass, more preferably within a range from 8 to 38% by mass, and most preferably within a range from 10 to 35% by mass with respect to the total amount of the cosmetic. It is preferable for the amount of the UV ray absorber which is blended in the (D) polar oil to be greater than or equal to 40 percent by mass, more preferably greater than or equal to 50 percent by mass, and still more preferably greater than or equal to 60 percent by mass. The entirety of the (D) polar oil may be a UV ray absorber. By the amount of the (D) polar oil being greater than or equal to 6 percent by mass, skin can be made to appear fuller and smoother. By the amount of the (D) polar oil being less than or equal to 40 percent by mass, a sensation of stickiness can be suppressed to a greater degree.

### (E) Cellulose Gum or Polysaccharide Thickener

It is preferable for the water in oil type emulsion cosmetic of the present disclosure to further contain (E) cellulose gum or a polysaccharide thickener. By the (E) cellulose gum or the polysaccharide thickener being contained, stability at high temperatures can be improved to a greater degree.

Cellulose gum (carboxy methyl cellulose sodium) is a cellulose series water soluble polymer. In the present disclosure, it is preferable for a cellulose gum having an average degree of polymerization within a range from about 150 to about 500 to be employed. It is possible to utilize a commercially available product such as Cellogen F-SR (by Daiichi Kogyo Seiyaku), for example.

Examples of the polysaccharide thickener include cellulose, hydroxyethyl cellulose, hydroxypropyl cellulose, methyl hydroxypropyl cellulose, methyl cellulose, quince seed, carrageenan, pectin, mannan, curdlan, chondroitin sulfate, starch, galactan, dermatane sulfate, glycogen, arabic gum, heparan sulfate, hyaluronic acid, sodium hyaluronate, tragant gum, keratan sulfate, chondroitin, xanthan gum, mucoitin sulfate, hydroxyethyl guar gum, carboxymethyl guar gum, guar gum, dextran, keratan sulfate, locust bean gum, succinoglucan, caronic acid, chitin, chitosan, carboxymethyl chitin, agar, etc., for example.

The amount of the (E) cellulose gum or polysaccharide thickener to be blended is preferably within a range from 0.05 to 2% by mass, and more preferably within a range from 0.1 to 1% by mass with respect to the total amount of the cosmetic. By the amount of the (E) cellulose gum or polysaccharide thickener being greater than or equal to 0.05% by mass, stability at high temperature can be improved to a greater degree. By the amount of the (E) cellulose gum or polysaccharide thickener being less than or equal to 2% by mass, a fresher sensation of use can be obtained.

The content of silicone oil in the water in oil type emulsion cosmetic of the present disclosure may be less than or equal to 10% by mass with respect to the total amount of the cosmetic, and the content of the dimethicone and/or cyclic silicone oil may be less than or equal to 10% by mass with respect to the total amount of the cosmetic. Even if the content of a silicone oil having favorable compatibility with the (A) cetyl PEG/PPG 10-1 dimethicone, and particularly the content of dimethicone and/or cyclic silicone oil is less than or equal to 10% by mass with respect to the total amount of the cosmetic, the water in oil type emulsion cosmetic of the present disclosure can exhibit favorable stability at high temperature due to the silicone was being blended therein.

An aqueous phase component of the water in oil type emulsion cosmetic of the present disclosure has water as a principal component and contains various water soluble components. It is preferable for the aqueous phase component to be blended in an amount within a range from 40 to 80% by mass, and more preferably within a range from 45% by mass to 70% by mass in the water in oil type emulsion cosmetic of the present disclosure. By the amount of the aqueous component being greater than or equal to 40% by mass, characteristics which are exhibited by containing water are more readily exhibited, and a fresh sensation of use can be obtained. By the amount of the aqueous component being less than or equal to 80% by mass, stability when placed in a high temperature environment can be improved.

One or a combination of two or more components which are generally employed in cosmetic and external pharmaceutical preparations, such as moisturizers, powder components, liquid oils and fats, higher fatty acids, higher alcohols, thickeners, saccharides, and medicaments may also be blended in the water based composition of the present disclosure. Components which may be blended in the water based composition of the present disclosure will be exemplified below.

Examples of moisturizers include, for example, propylene glycol, glycerin, 1, 3-butylene glycol, xylitol, sorbitol, maltitol, chondroitin sulfate, hyaluronic acid, mucoitin sulfate, caloninic acid, atelocollagen, sodium lactate, bile salt, d, l-pyrrolidone carboxylate salt, short chain soluble collagen, diglycerin (EO) PO adduct, chestnut rose extract, bupleurum extract, melilot extract, etc.

Examples of the powder component include an inorganic powder (for example, talc, kaolin, mica, sericite, muscovite, phlogopite, synthetic mica, red mica, biotite, vermiculite, magnesium carbonate, calcium carbonate, aluminum silicate, barium silicate, calcium silicate, magnesium silicate, strontium silicate, tungstenate metal salt, magnesium, silica, zeolite, barium sulfate, calcined calcium sulfate (baked gypsum) calcium phosphate, fluorine apatite, hydroxy apatite, ceramic powder, metal soap (for example, zinc myristate, calcium palmitate, and aluminum stearate), boron nitride, etc.); an organic powder (for example, polyamide resin powder (nylon powder), polyethylene powder, methyl polymethacrylate powder, polystyrene powder, a styrene-acrylate copolymer resin powder, benzo quanamine resin powder, poly tetra fluorinated ethylene powder, cellulose powder, etc.); an inorganic white pigment (for example, titanium dioxide, zinc oxide, etc.); an inorganic red type pigment (for example, iron oxide, iron titanate, etc.); an inorganic purple type pigment (for example, manganese violet, cobalt violet, etc.); an inorganic green pigment (for example, chromium oxide, chromium hydroxide, cobalt titanate, etc.); an inorganic blue type pigment (for example, ultramarine, Prussian blue, etc.); a pearl pigment (for example, titanium oxide coated mica, titanium oxide coated bismuth oxychloride, titanium oxide coated talc, colored titanium oxide coated mica, bismuth oxychloride, fish scale foil, etc.); a metal powder pigment (for example, aluminum powder, copper powder, etc.); an organic pigment such as zirconium, barium, and aluminum flakes (for example, organic pigments such as red number 201, red number 202, red number 204, red number 205, red number 220, red number 226, red number 228, red number 405, orange number 203, orange number 204, yellow number 205, yellow number 401, and blue number 404, red number 3, red number 104, red number 106, red number 227, red number 230, red number 401, red number 505, orange number 205, yellow number 4, yellow number 5, yellow number 202, yellow number 203, green number 3, and blue number 1); natural color (for example, chlorophyll, β-carotene, etc.); etc.

Examples of liquid oils and fats include, for example, avocado oil, camellia oil, turtle oil, macadamia nut oil, corn oil, mink oil, olive oil, rapeseed oil, egg yolk oil, sesame oil, persic oil, wheat germ oil, camellia sasanqua oil, castor oil, linseed oil, safflower oil, cottonseed oil, perilla seed oil, soybean oil, peanut oil, tea seed oil, kaya oil, rice bran oil, cinnamon oil, Japanese Paulonia oil, jojoba oil, germ oil, triglycerin, etc.

Examples of the higher fatty acids include, for example, lauric acid, myristic acid, palmitic acid, stearic acid, behenic acid, oleic acid, undecylenic acid, tallic acid, isostearic acid, linoleic acid, linolenic acid, eicosapentaenoic acid (EPA), docosahexaenoic acid (DHA), etc.

Examples of polyhydric alcohols include, for example, straight chain alcohols (such as lauryl alcohol, cetyl alcohol, stearyl alcohol, behenyl alcohol, myristyl alcohol, oleyl alcohol, and cetostearyl alcohol); and branched chain alcohols (such as monostearyl glycerin ether (batyl alcohol), 2-decyltetradecinol, lanolin alcohol, cholesterol, phytosterol, hexyldodecanol, isostearyl alcohol, and octyldodecanol), etc.

A thickener other than the (E) component may also be blended. Specific examples include plant based polymers such as carob gum, karaya gum, agar, algae colloid (brown algae extract), microorganism based polymers such as pullulan, animal based polymers such as collagen, casein, albumin, and gelatin, and alginate series polymers such as sodium alginate and propylene glycol ester alginate, etc.

Additional examples of a thickener include vinyl based polymers such as polyvinyl methyl ether and carboxy vinyl polymer, poly oxyethylene based polymers, poly oxyethylene poly oxy propylene copolymer based polymers, acrylic polymers such as poly ethyl acrylate and polyacrylamide, inorganic aqueous polymers such as polyethylene imine, cationic polymers, bentonite, magnesium aluminum silicate, laponite, hectorite, and silicic anhydride, PEG-240/decyltetradeceth-20/hexamethylene diisocyanate copolymer, (dimethyl lacrylamide/acryloyl dimethyl taurine Na)) cross polymer, (Na acrylate/acryloyl dimethyl taurine) copolymer, (alkyl acrylate/steares methacrylate-20) copolymer, (acryloyl dimethyl taurine ammonium/VP) copolymer, etc., dextrin, sodium pectinate, sodium alginate, cellulose dialkyl dimethyl ammonium sulfate, aluminum magnesium silicate, bentonite, hectorite, AlMg silicate (bee gum), laponite, silicate anhydride, etc.

Examples of monosaccharides include, for example, trioses (D-glyceryl aldehyde, dihydroxyacetone, etc., for example); tetracarboses (eg, D-erythrose, D-erythrose, D-treose, etc.); pentoses (L-arabinose, D-xylose, L-lyxose, D-arabinose, D-ribose, D-ribrose, D-xylrose, L-xylrose, etc., for example); hexoses (D-glucose, D-talose, D-busicose, D-galactose, D-fructose, L-galactose, L-mannose, D-tagatose, etc., for example); heptoses (aldoheptose, heplose, etc., for example); octoses (octulose, etc., for example); deoxy sugars (2-deoxy-D-ribose, 6-deoxy-L-galactose, 6-deoxy-L-mannose, etc., for example); amino sugars (D-glucosamine, D-galactosamine, sialic acid, amino uronic acid, muramic acid, etc., for example); uronic acids (D-glucuronic acid, D-mannuronic acid, L-glulonic acid, D-galacturonic acid, L-isulonic acid, etc., for example).

Examples of oligosaccharides include, for example, sucrose, guntianose, umbelliferose, lactose, planteose, isolicnoses, α, α-trehalose, raffinose, lycnoses, umbilicin, stachyose velvascose, etc.

Examples of amino acids include, for example, neutral amino acids (threonine, cysteine, etc., for example); basic amino acids (hydroxylysine, etc., for example), etc. Examples of amino acid derivatives include, for example, sodium acyl sarcosine (sodium lauroyl sarcosine), acyl glutamate, sodium acyl β-alanine, glutathione, pyrrolidone carboxylic acid, etc.

Examples of organic amines include, for example, mono ethanolamine, diethanolamine, triethanolamine, morpholine, tri iso propanolamine, 2-amino-2-methyl-1,3-propanediol, 2-amino-2-methyl-1-propanol. etc.

Examples of alkylene oxide derivatives include, for example, POE (9) POP (2) dimethyl ether, POE (14) POP (7) dimethyl ether, POE (10) POP (10) dimethyl ether, POE (6) POP (14) dimethyl ether, POE (15) POP (5) dimethyl ether, POE (25) POP (25) dimethyl ether, POE (7) POP (12) dimethyl ether, POE (22) POP (40) dimethyl ether, POE (35) POP (40) dimethyl ether, POE (50) POP (40) dimethyl ether, POE (55) POP (30) dimethyl ether, POE (30) POP (34) dimethyl ether, POE (25) POP (30) dimethyl ether, POE (27) POP (14) dimethyl ether, POE (55) POP (28) dimethyl ether, POE (36) POP (41) dimethyl ether, POE (7) POP (12) dimethyl ether, POE (17) POP (4) dimethyl ether, etc.

Examples of metal ion sequestering agents include, for example, 1-hydroxyethane-1,1-diphosphonic acid, 1-hydroxyethane-1,1-diphosphonic acid tetrasodium salt, disodium edetate, tri sodium edetate, tetrasodium edetate, sodium citrate, sodium polyphosphate, sodium metaphosphate, gluconic acid, phosphoric acid, citrate, ascorbic acid, succinic acid, edetic acid, trisodium ethylene diamine hydroxyethyl triacetate, etc.

Examples of antioxidizing aiding agents include phosphoric acid, citric acid, ascorbic acid, maleic acid, malonic acid, succinic acid, fumaric acid, kephalin, hexametaphosphate, phytic acid, ethylenediamine tetraacetic acid, etc.

Examples of oil soluble medicaments include oil soluble vitamins such as vitamin A (retinol), vitamin D, vitamin E, vitamin K, and derivatives thereof (vitamin A oil, retinol palmitate, etc.), oil soluble derivatives of water soluble medicaments such as vitamin C and arbutin (vitamin palmitate, etc.), oil soluble plant extracts, oil soluble fragrances, substances having surfaces which have been hydrophobized, cyclosporin, etc., for example.

Other components which may be blended include, for example, preservatives (ethyl paraben, butylparaben, etc.); whitening agents (placenta extract, saxifrage extract, arbutin, etc., for example); blood circulation promoters (nicotinic acid, benzyl nicotinate, tocopherol nicotinate, β-butoxy ester nicotinate, minoxidyl or its relatives, γ-orizanol, alkoxy carbonyl pyridine N-oxide, carpronium chloride, acetylcholine or derivatives thereof, etc.); various extracts (ginger, phellodendron, coptis, lithospermum, birch, loquat, carrot, aloe, mallow, iris, grape, loofah, lily, saffron, cnidium, zingiber, hypericum, ononis, garlic, capsicum, citrus peel, Japanese angelica, peony, seaweed, etc., for example); activator agents (pantenyl ethyl ether, nicotinic acid amide, biotin, pantothenic acid, royal jelly, cholesterol derivatives, etc., for example); antilipolytic agents (pyridoxins, thiantoll, etc., for example), etc.

Specific product applications for the water in oil type emulsion cosmetic of the present disclosure include milky liquids and creamy products such as a milky lotion, a skin care cream, a hair cream, a liquid foundation, an eyeliner, mascara, and an eye shadow. These products are produced by standard methods with the components described above.

### EXAMPLES

The present disclosure will be described in greater detail with reference to the following Examples. However, the present disclosure is not limited to these Examples. In addition, the amounts of the components which are blended in the Examples are represented by percentages by mass unless otherwise noted.

Water in oil type emulsion cosmetics having the compositions shown in Table 1 below were prepared by standard methods and evaluations were conducted according to the following standards.

### (Emulsion Stability)

The viscosities of the prepared water in oil type emulsion cosmetics were measured twice, once on the day that they were prepared and once after being stored for one week at 50° C. Emulsion stability was evaluated by the percentage of decrease in viscosity. The viscosities were measured at 30° C. with a BL type viscometer (Model VS-A by Shibaura Systems) that stirred the cosmetics with Rotor Number 4, 12 rpm for one minute. Emulsion stability was judged to be poor in cases that the viscosity decreased by greater than or equal to 10 percent (less than or equal to 0.9).

### (Smoothness)

The prepared water in oil type emulsion cosmetics were used by a panel of 10 experts, and evaluated according to the following criteria.

### <Evaluation Criteria>

A: 9 or more panelists felt that the cosmetic was smooth
B: 6 to 8 panelists felt that the cosmetic was smooth
C: 5 or fewer panelists felt that the cosmetic was smooth

### (Freshness)

The prepared water in oil type emulsion cosmetics were used by a panel of 10 experts, and evaluated according to the following criteria.

### <Evaluation Criteria>

A: 9 or more panelists felt that the cosmetic imparted a sensation of freshness
B: 6 to 8 panelists felt that the cosmetic imparted a sensation of freshness
C: 5 or fewer panelists felt that the cosmetic imparted a sensation of freshness

Note that the details of the product names of the raw materials in the table are as follows.

### *1: ABIL EM180

### *2: ABIL EM90

*3: Silicone Wax AMS-C30 (melting point: 70° C.)
*4: Hydrocarbon Wax (melting point: 68 to 72° C.)
*5: Sugar Wax A-10E (melting point: 44 to 50° C.)
*6: Polyethylene Glycol (molecular weight: 6000)

TABLE 1

| | | Example 1 | Example 2 | Example 3 | Example 4 |
|---|---|---|---|---|---|
| (A) | Cetyl PEG/PPG 10-1 | 1.5 | 1.5 | 1.5 | 1.5 |
| | dimethicone (*1) | | | | |
| | Cetyl PEG/PPG 10-1 | 1.5 | 1.5 | 1.5 | 1.5 |
| | dimethicone (*2) | | | | |
| (D) | Ethyl hexyl salicylate | 5 | 5 | 5 | 5 |
| | Ethyl hexyl methoxy | 5 | 5 | 5 | 5 |
| | cinnamate | | | | |
| | Diethyl amino hydroxy | 1 | 1 | 1 | 1 |
| | benzoyl hexyl benzoate | | | | |
| | Diisopropyl sebacate | 2 | 2 | 2 | 2 |
| | PPG-17 | 1 | 1 | 1 | 1 |
| | Dimethicone | 4 | 4 | 4 | 4 |
| | Caprylyl Methicone | 4 | 4 | 4 | 4 |
| Wax | Alkyl (C30-45) methicone, | 0.7 | 2 | 0.7 | 0.7 |
| | olefin (C30-45) (*3) | | | | |
| | Candellila wax (*4) | | | | |
| | Sucrose tetrastearate | | | | |
| | triacetate (*5) | | | | |
| | Polymethyl silsesquioxane | 5 | 5 | 5 | 5 |
| | Water | 51.5 | 50.2 | 51.7 | 50.5 |
| | DPG | 2 | 2 | 2 | 2 |
| (E) | Cellulose Gum | 0.2 | 0.2 | | 0.2 |
| | Ethanol | 8 | 8 | 8 | 8 |
| | Phenoxy Ethanol | 0.5 | 0.5 | 0.5 | 0.5 |
| | Glycerin | 5 | 5 | 5 | 5 |
| (C) | PEG-150 (*6) | 1 | 1 | 1 | 2 |
| | Sodium Chloride | 0.9 | 0.9 | 0.9 | 0.9 |
| | EDTA-3Na | 0.2 | 0.2 | 0.2 | 0.2 |
| Total | | 100 | 100 | 100 | 100 |
| Properties | Viscosity on Day of | 18480 | 32340 | 19100 | 17580 |
| | Preparation (mPa·s) | | | | |
| | Viscosity after 1 week | 17950 | 34200 | 17850 | 17330 |
| | at 50° C. (mPa·s) | | | | |
| | Decrease in Viscosity | 0.971 | 1.058 | 0.935 | 0.986 |
| | (Viscosity after 1 week | | | | |
| | at 50° C./Viscosity on | | | | |
| | Day of Preparation) | | | | |
| Usability | Smoothness | A | B | A | A |
| | Freshness | A | A | A | B |
| | | Comparative | Comparative | Comparative | Comparative |
| | | Example 1 | Example 2 | Example 3 | Example 4 |
| (A) | Cetyl PEG/PPG 10-1 | 1.5 | 1.5 | 1.5 | 1.5 |
| | dimethicone (*1) | | | | |
| | Cetyl PEG/PPG 10-1 | 1.5 | 1.5 | 1.5 | 1.5 |
| | dimethicone (*2) | | | | |
| (D) | Ethyl hexyl salicylate | 5 | 5 | 5 | 5 |
| | Ethyl hexyl methoxy | 5 | 5 | 5 | 5 |
| | cinnamate | | | | |
| | Diethyl amino hydroxy | 1 | 1 | 1 | 1 |
| | benzoyl hexyl benzoate | | | | |
| | Diisopropyl sebacate | 2 | 2 | 2 | 2 |
| | PPG-17 | 1 | 1 | 1 | 1 |
| | Dimethicone | 4 | 4 | 4 | 4 |
| | Caprylyl Methicone | 4 | 4 | 4 | 4 |
| Wax | Alkyl (C30-45) methicone, | | 0.7 | | |
| | olefin (C30-45) (*3) | | | | |
| | Candellila wax (*4) | | | 0.7 | |
| | Sucrose tetrastearate | | | | 0.7 |
| | triacetate (*5) | | | | |
| | Polymethyl silsesquioxane | 5 | 5 | 5 | 5 |
| | Water | 52.2 | 52.5 | 51.5 | 51.5 |
| | DPG | 2 | 2 | 2 | 2 |
| (E) | Cellulose Gum | 0.2 | 0.2 | 0.2 | 0.2 |
| | Ethanol | 8 | 8 | 8 | 8 |
| | Phenoxy Ethanol | 0.5 | 0.5 | 0.5 | 0.5 |
| | Glycerin | 5 | 5 | 5 | 5 |
| (C) | PEG-150 (*6) | 1 | | 1 | 1 |
| | Sodium Chloride | 0.9 | 0.9 | 0.9 | 0.9 |
| | EDTA-3Na | 0.2 | 0.2 | 0.2 | 0.2 |
| Total | | 100 | 100 | 100 | 100 |
| Properties | Viscosity on Day of | 12100 | 18350 | 11010 | 14650 |
| | Preparation (mPa·s) | | | | |
| | Viscosity after 1 week | 9680 | 16310 | Precipitation | 12270 |
| | at 50° C. (mPa·s) | | | | |
| | Decrease in Viscosity | 0.800 | 0.889 | — | 0.838 |
| | (Viscosity after 1 week | | | | |
| | at 50° C./Viscosity on | | | | |
| | Day of Preparation) | | | | |
| Usability | Smoothness | A | A | C | B |
| | Freshness | A | A | C | B |

As shown in Table 1, all of the water in oil type emulsion cosmetics of the present disclosure were smooth, exhibited a fresh sensation of use, and had superior emulsion stability. In contrast, Comparative Example 1 which did not contain wax and Comparative Example 2 which did not contain polyethylene glycol did not achieve emulsion stability. In Comparative Example 3 which employed a hydrocarbon wax, wax crystals precipitated during severe testing, viscosity measurement could not be conducted after one week for Comparative Example 3, and usability was also poor. Comparative Example 4 which employed sugar wax also exhibited poor emulsion stability.

1. A water in oil type emulsion cosmetic comprising:
(A) cetyl PEG/PPG 10-1 dimethicone;
(B) silicone wax;
(C) polyethylene glycol; and
(D) a polar oil.

2. The water in oil type emulsion cosmetic according to claim 1, wherein: the (D) polar oil is contained in an amount within a range from 6 to 40% by mass with respect to the total amount of the cosmetic.

3. The water in oil type emulsion cosmetic according to claim 1, wherein:
the molecular weight of the (C) polyethylene glycol is within a range from 6000 to 20000.

4. The water in oil type emulsion cosmetic according to claim 1, further comprising:
(E) one of cellulose gum and a polysaccharide thickener.

5. The water in oil type emulsion cosmetic according to claim 1, wherein:
the content of dimethicone and/or cyclic silicone oil is less than or equal to 10% by mass with respect to the total amount of the cosmetic.
